(19) United States
(12) Patent Application Publication
(10) Pub. No.: US 2024/0094196 A1
(43) Pub. Date: Mar. 21, 2024

(54) In-Line Testers with Colorimetric Test Chambers and a Detachable Reservoir

(71) Applicant: Nasogastric Feeding Solutions Ltd, Liverpool Merseyside (GB)

(72) Inventors: Robert Senior, Liverpool Merseyside (GB); George Gallagher, Liverpool Merseyside (GB)

(21) Appl. No.: 18/261,963

(22) PCT Filed: Jan. 18, 2022
(86) PCT No.: PCT/GB2022/050132

§ 371 Date: Jul. 18, 2023

(30) Foreign Application Priority Data
Jan. 19, 2021 (GB) 2100703.4

(51) Int. Cl. G01N 33/52; A61J 15/00; G01N 21/80

(52) U.S. Cl. CPC G01N 33/528; A61J 15/0084; A61J 15/0092; G01N 21/80; A61J 2200/70

(57) ABSTRACT

A tester includes: an inlet, connectable to an aspirator tube; an outlet connectable to a vacuum source; and a chamber between the inlet and the outlet, the chamber including: a first tester arranged such that fluids drawn into the chamber by the vacuum source come into contact therewith; a second tester arranged such that fluids drawn into the chamber by the vacuum source pass through it; and a reservoir between the first tester and the second tester; where the first tester includes a first substance adapted to change color in the presence of a target substance, and where the second tester includes colorimetric capnometer, which exhibits a color change in the presence of carbon dioxide, the in-line tester further including a liquid-stop device which, when dry, permits passage of gas from inlet to outlet, but when wetted by liquid, inhibits or prevents the passage of fluids from inlet to outlet.

### CROSS REFERENCE TO RELATED APPLICATIONS

This application is a U.S. national phase of International Application No. PCT/GB2022/050132 filed Jan. 18, 2022, which claims priority to United Kingdom Application No. 2100703.4 filed Jan. 19, 2021, all of which are incorporated by reference herein in their entirety.

### FIELD OF DISCLOSURE

This invention relates to in-line testers, and in particular, but without limitation, to in-line testers that can be used in conjunction with a nasogastric (hereinafter “NG”) aspirator to test for the correct placement of an NG tube.

### BACKGROUND

Aspirators are used in a wide range of medical procedures where fluids need to be drawn from within a body cavity, for example, for diagnostic, sampling and/or therapeutic purposes.

An aspirator generally comprises a vacuum source connected to a tube that can be inserted, or fed, into a body cavity. The vacuum source can be of any suitable type, such as a syringe; a syringe-based manual pump (such as that disclosed in published UK patent No: GB2523591B); or an electric vacuum pump (such as that described in published UK patent application No GB2547012).

When the vacuum is applied to the free end of the tube, the vacuum draws fluid from the body cavity through the tube, provided, of course, that the tip of the tube is located within the fluid to be aspirated. A liquid trap is usually interposed between the tube and the vacuum source to prevent aspirated liquids from being drawn into the vacuum source, thereby reducing the likelihood of damaging and/or contaminating the vacuum source.

One example of a liquid trap is disclosed in published UK patent No: GB2523591B, in which a porous or perforated membrane is used to allow gasses to pass through it, but when wetted by aspirated liquids, blocks the aspirate from passing through it.

When using an aspirator, care needs to be taken to ensure that the vacuum is not too high and that the quantity and rate of aspiration is monitored. Monitoring and control circuitry can often be used to facilitate this, as well as the manual interventions of an experienced operator.

It is commonplace to use aspirators to assist in determining whether the tip of a NG feeding tube is correctly positioned. When feeding a patient using a NG feeding tube, care must be taken to ensure that the tip of the NG tube is positioned within the patient's stomach, rather than in his/her lungs. The reason that this is important is that a NG tube is fed into the patient's throat via his/her nose, and due to the bifurcation of the patient's throat into the oesophagus and trachea, it is possible for the tip of the NG tube to be fed into the trachea, rather than the oesophagus (i.e. the wrong way) resulting in the tip of the NG tube being positioned within the patient's lung. If feeding commences with the NG tube positioned in the patient's lung, the results can be very serious.

As such, medical protocols require that before NG feeding commences, correct positioning of the NG feeding tube tip is determined. The only definitive way to determine the correct placement of a NG feeding tube is via a chest X-ray or other imaging procedure. However, chest x-rays have been found, in certain cases, to be less than definitive because the angle of exposure, level of exposure, patient position and the skill of the radiographer are all important in achieving an diagnostic x-ray image that is fit for purpose. Further, the time taken for an x-ray to be booked, carried out, processed and reported can vary considerably—during which time a subject is denied feeding, the subject most likely being in a critical condition. Additionally, for neonates particularly, the subject will be exposed to radiation with the associated potentially negative consequences.

Another way to check for the correct positioning of an NG tube is to aspirate and test a sample of fluid via the NG tube prior to feeding. This is indeed the current clinical standard in the UK, whereby fluids are aspirated via the NG tube and are checked for acidity using pH paper. A colour change to indicate acidic pH is considered to be an indicator that the NG tube is in the correct location (it being assumed that the stomach contents are acidic, whereas lung fluids are not) and so feeding may commence.

If, however, a basic (alkali or pH>5.5) pH reading is obtained, then the assumption is that the location of the tube is not in the stomach and the NG tube will be withdrawn and reinserted before a further pH test is carried out.

However, pH testing has been found to be unreliable insofar as it can yield false negative results, for example, if the patient is taking antacids to prevent reflux. In this case, irrespective of whether the NG tube is in the stomach or not, a basic or neutral pH will be indicated from any sample obtained using the current clinical standard.

Therefore, the current guidelines stipulate that if an acidic pH reading is obtained, then it is safe to commence feeding, whereas if a basic (alkali) pH reading is obtained, further investigation is required. The basic “rule of thumb” is therefore: acid=feed; alkali/neutral=do not feed.

However, current pH testing protocols fail to take into account the possibility of false positive results (i.e. acid, but not safe to feed), which can prove problematic to patient health, or fatal in extreme cases. False positives can occur when the NG tube tip is located in the lung of a patient, but where the patient is suffering from reflux resulting in some gastric content being inhaled/present in the lung. Clearly, in this case, it is possible for the pH of an aspirated sample to be acidic—indicating, according to current clinical protocols, that the NG tube tip is in the correct location, i.e. in the stomach, and so feeding will commence. However, in this example, the NG tube would not be correctly located, and the consequences of commencing feeding could be very serious.

It will be appreciated that the aforesaid protocols, devices and methods are not ideal, and a need therefore exists for an improved and/or an alternative protocol, device and/or method.

### SUMMARY

Various aspects of the invention are set forth in the appended claims.

According to a first aspect of the invention, there is provided an in-line tester comprising main body, an inlet connectable in use to an NG tube, an outlet, connectable in use to a vacuum source, an exhaust port in fluid communication with air surrounding the main body and a fluid passageway interconnecting the inlet and the outlet, the in-line tester additionally comprising, and each of the following being connected in-line with the fluid passageway: first and second chambers each containing a colorimetric tester; a first one-way valve in-line with the fluid passageway permitting the flow of fluids from the inlet to the outlet; a liquid-stop device interposed between the first and second chambers that permits the flow of gasses, but which inhibits or prevents the flow of liquids, towards the second chamber; and a reservoir interposed between the first and second chambers; the in-line tester further comprising: a second one-way valve permitting the flow of fluids from the outlet to the exhaust port; and the in-line tester being characterised by: the reservoir comprising a container that is detachably connected to the main body; and the main body comprising an attachment formation comprising: a seal that sealingly and detachably connects the container to the main body, a first port connecting the fluid passageway upstream of the attachment formation to the interior of the container; and a second port connecting the fluid passageway downstream of the attachment formation to the interior of the container.

The provision of a reservoir enables the progress of an aspiration procedure to be monitored. It also addresses the problem where the NG tube contains a flush liquid that needs to be aspirated before the stomach contents is aspirated and tested. The invention is distinguished over the prior art by virtue of the reservoir being detachable from the main body of the in-line tester, which permits offline or after-the-event analysis to be carried out. It also enables the reservoir to be emptied and/or cleaned mid-aspiration.

Suitably, the fluid passageway interconnecting the inlet and the first chamber comprises a loft, which transitions from a substantially circular cross-section to a substantially rectangular cross-section. In the context of the present disclosure, the term “loft” means a feature that blends and transitions multiple profiles into smooth shapes. The rectangular cross-sectioned part of the loft suitably aligns with a leading edge of the colorimetric capnometer of the first chamber. By providing a loft between the inlet and the first chamber, fluids, and in particular liquids, entering the in-line tester can be given a flow profile that converts the substantially cylindrical flow entering the in-line tester via an NG tube attached to the inlet into a flat sheet of fluid/liquid that advances with a substantially linear front across a surface, and in particular, a rear surface of the colorimetric tester of the first chamber. Suitably, the interior volume of the inlet, loft and space within the first chamber behind the colorimetric tester is less than substantially 0.25 ml, and this enables an extremely small volume of aspirate to be collected and still contact the colorimetric tester within the first chamber. In order to obtain a substantially linear flow path across the back of the colorimetric tester of the first chamber, the lot suitably comprises a translation component that is perpendicular to an access of the inlet of the in-line tester. In other words, the loft suitably comprises a “swan neck”, which serves to shape the liquid front and spread it laterally so as to form the line front that passes over a rear surface of the colorimetric tester.

The attachment formation suitably comprises an attachment body extending from the main body, the first and second ports being formed as through holes in the attachment body. In one embodiment, the attachment body is substantially cylindrical or a truncated cone, which can be press-fitted onto a tubular neck of the detachable reservoir.

The seal suitably comprises an elastomeric member located around the attachment formation, which, in certain embodiments, allows it to provide a dual function of sealing with, and frictionally gripping, the container. The sheath is suitably manufactured of silicone rubber, which is hypoallergic and easily cleaned.

Preferably, the seal comprises an elastomeric member that is over-moulded onto the attachment formation. Over-moulding suitably prevents inadvertent separation of the elastomeric member from the from attachment formation.

Most preferably, the elastomeric member comprises a ribbed outer surface profile, the ribs forming one or more compressible sealing rings that friction-fit and seal to a complementary inner surface of the container.

The liquid-stop device is suitably located on the attachment body and sealingly covers the second port. This configuration permits liquids to be collected in the reservoir but inhibits or prevents the egress of the aspirated liquid beyond the reservoir. In certain embodiments, the liquid-stop device comprises a labyrinthine liquid trap, such as a gooseneck. In preferred embodiments of the invention, the liquid-stop device comprises a porous or perforated element whose pores are sized so as to permit the passage of gasses therethrough, but which inhibit or prevent the passage of liquids therethrough. This can be accomplished by forming the liquid-stop device from a web of perforated polymer, or by manufacturing the liquid-stop device from a web of porous paper or cardboard.

Preferably, a further liquid-stop device is provided that is interposed between the colorimetric capnometer of the second chamber and the outlet of the in-line tester. The purpose of this additional liquid-stop device is to prevent liquids being introduced into the in-line tester inadvertently and coming into contact with the colorimetric capnometer. It is well-known that a colorimetric capnometer, once wetted, cannot be used again, and so the provision of a secondary liquid-stop device downstream of the colorimetric capnometer serves to ensure that the colorimetric capnometer remains dry regardless of whether liquids are introduced via the inlet or the outlet of the in-line tester. For example, where a syringe has been flushed, it may contain droplets of liquid. Without a colorimetric capnometer present between the outlet of the in-line tester and the colorimetric capnometer, those liquid droplets within the syringe could easily be pushed into the in-line tester and cause the colorimetric capnometer to become wetted. By providing a secondary liquid-stop device between the colorimetric capnometer and the outlet, the likelihood of this occurring is greatly minimized.

The liquid-stop device suitably comprises a porous of perforated element, which is at least partially manufactured from, or coated with, a hydrophobic material; or which has a hydrophobic layer on it. The function of such a liquid-stop device is essentially that; when it is dry, gasses (including air) are able to pass through the pores or perforations therein, thus permitting the passage or transduction of gases/air through the liquid-stop device. However, when the liquid-stop device is wetted by a liquid, in this case an aspirated liquid, the hydrophobicity of the liquid-stop device repels the liquid from its surface—towards the pores/perforations, which are not hydrophobic. This results in liquid preferentially covering the liquid-stop device's pores/perforations, thereby preventing gas/air from passing through it. As such, when the liquid-stop device is dry, for example, during the initial stage of an aspiration, where air is primarily drawn up through the NG tube, the aspirated air is able to pass through the liquid-stop device. However, subsequently, when liquid is aspirated, when that liquid eventually reaches the liquid-stop device, it will wet it, thereby preventing any further passage of fluid (that is to say liquids or gases) through the liquid-stop device.

The location of the liquid-stop device between the first and second chambers is particularly beneficial in NG aspirator applications, where gasses (air/gas from the stomach/lung) are usually aspirated before liquids. Thus, by placing the colorimetric capnometer downstream of the liquid-stop device and before the outlet, sequential testing may be possible. Specifically, when the in-line tester of the invention is used in conjunction with an NG aspirator, typically gasses will be aspirated before liquids are drawn-up through the NG tube. Initially, the liquid-stop device will be dry, which enables the aspirated gasses to pass through the first chamber and the reservoir before coming into contact with the colorimetric capnometer, and so test for carbon dioxide in the aspirate. Later, liquids may then be aspirated, and when this happens, the liquids come into contact with the first colorimetric tester, and are tested for the target substance thereby. When, eventually, the liquid-stop device is wetted by an aspirated liquid, it inhibits or prevents the passage of liquids through it, thereby keeping the colorimetric capnometer dry.

Preferably, the container is manufactured of a transparent material, such as glass or plastics, as this permits the aspirate to be visually inspected. The container may have volumetric graduations to permit the volume of aspirate to be measured or estimated.

In certain embodiments of the invention, the outlet is connectable, in use, to a syringe. The provision of the one-way valves means that when the syringe plunger is withdrawn, aspirate is drawn up through the NG tube as the first one-way valve opens and the second one-way valve closes. However, when the syringe plunger is depressed, fluid (air) within the syringe is vented via the vent port because the first one-way valve closes and the second one-way valve opens. The second one-way vale is suitably located in a branch off the main fluid passageway.

To facilitate connection of the in-line tester to an NG tube and/or a syringe or other vacuum source, the inlet and/or outlet suitably comprise any one of more of: a luer; a luer-lock; a screw thread; a bayonet connector; and a tapered nipple. Suitably, the inlet comprises a connector for connecting the inlet to the connector of enteral or NG tube, such as a female Luer or female Luer-lock connector. Suitably, the outlet comprises a connector suitable for connecting it to an enteral syringe, such as a male Luer or male Luer-lock connector.

Preferably, the colorimetric tester of the first chamber exhibits a colour change in response to the pH of a fluid in contact therewith. The colorimetric tester of the first chamber could be litmus paper or pH indicator paper. Testing for the acidity/alkalinity of the aspirate enables a user to gauge whether or not the tip of the NG tube is located within stomach acid.

Preferably, the colorimetric tester of the second chamber comprises a colorimetric capnometer, which exhibits a colour change in response to the concentration of carbon dioxide in contact therewith. This permits a user to gauge whether or not the tip of the NG tube is located within the breathing passageways or lungs of a patient.

By providing both pH and CO2 testers, it is possible to determine correct/incorrect placement of the NG tube tip in the stomach/lung, respectively.

For simplicity, the either or both of the one-way valves suitably comprise diaphragm check valves.

The main body suitably comprises a set of stacked components, the stack comprising an upper component, a lower component and one or more intermediate components interposed between the upper and lower components. The fluid passageway can be formed by a recess formed in one component of the stack, the open part of the recess being closed by a surface of an adjacent component of the stack. Additionally or alternatively, the fluid passageway may be formed by complementary recesses formed in adjacent components of the stack, the open parts of the recesses aligning so as to form the channel intersecting the interface between the adjacent layers. Additionally or alternatively, the fluid passageway may be formed by a through hole in one component of the stack, which aligns with a recess or surface of adjacent components of the stack on either side.

The first and second chambers suitably comprise windows, which permit visual inspection of the colorimetric testers. The windows may be formed by the upper component of the stack being manufactured from a transparent material and by one or more chambers being formed by a cavity formed in an adjacent component of the stack; or by a through-aperture in a first adjacent component of the stack and by a surface aligning with the through-aperture second component of the stack adjacent the first adjacent component of the stack.

In one possible embodiment of the invention, the liquid-stop device comprises a porous or perforated element, which separates the inlet from the outlet at some point along the fluid flow path. Therefore, fluids (liquid or gases) drawn into the in-line tester must come into contact with the porous or perforated element at some point. In one possible embodiment, the liquid-stop device comprises a small piece of pH indicator paper with a hydrophobic plastics backing layer. Thus, liquids drawn into the in-line tester can be tested for pH by the pH paper, but when the pH paper is wetted, the liquid also wets the hydrophobic layer behind it, thereby closing-off the fluid pathway. In other embodiments, the pH paper can be substituted for capnometry indicator paper, which changes colour in the presence, or otherwise, of carbon dioxide.

Suitably, a one-way valve is provided at, or downstream of the second chamber. Thus, a sample of aspirated gas can be trapped/retained within the second chamber between the (now wetted) liquid-stop device and the one-way valve. This is particularly beneficial when using colorimetric capnometry because colorimetric capnometers tend to revert to their initial colour after a relatively short period of time. However, by effectively trapping a sample of aspirated gas in the chamber between the liquid-stop device and a one-way check valve, reversion of the colour of the colorimetric capnometer back to its initial state is slowed or inhibited.

As previously mentioned, the colorimetric tester of the first chamber is suitably adapted to exhibit a colour change in the presence of acid such as gastric juice found in the stomach of a patient. The acid can be stomach acid (e.g. HCl), in which case, the first tester may comprise, e.g. on a liquid-absorbent layer thereof, a colorimetric substance that is an acid-base indicator, such as litmus paper or paper comprising Bromothymol sulfonephthalein.

Additionally or alternatively, the colorimetric tester of the first chamber can be used to test for a stomach-related marker, which may comprise any compound or biological structure, such as a cell or a cell fragment, an enzyme, a chemical etc., which is found within the stomach of a patient, but preferably not the lung of a patient. In certain embodiments of the invention, the stomach-related marker may comprise any one or more of the group comprising: gastric enzyme (or substrate thereof); gastric hormone; pepsin; pepsinogen; intrinsic factor (IF); vitamin B12-IF complex; mucin; gastrin; gastric lipase; and trypsin. In an embodiment, the in-line tester comprises detection means for gastric lipase. The detection means for gastric lipase may comprise tributyrin. The in-line tester may contain means for detecting the presence of two or more stomach-related compounds. Advantageously, the device comprises means for detecting two stomach-related markers.

Suitably, therefore, the in-line tester may comprise a further tester having a further colorimetric substance adapted to exhibit a colour change in the presence of a second target substance. In certain embodiments, the first and further testers may be incorporated into a single device, that is to say being is divided into discrete regions, each discrete region comprising a different colorimetric substance adapted to exhibit a colour change in the presence of different target substances.

The colorimetric capnometer is adapted to detect carbon dioxide, and this may be by using a specially adapted form of indicator paper, impregnated with a dye that changes colour from, say, purple to yellow in the presence of carbon dioxide. Carbon dioxide monitoring to check NG tube position has been suggested (Thomas and Falcone, J Am Coll Nutr 1998, 17(2):195-7). Various trials have used either capnography (direct carbon dioxide measurement) or colorimetric capnometry (based on colour change of adapted pH paper with sulfonephthalein). It has already been shown that that there is a higher concentration of carbon dioxide in exhaled air from the lungs compared to any air obtained from a gastric aspirate. However, the use of measuring carbon dioxide provides no information about tube placement within the gastrointestinal tract and administration of enteral nutrition may be delivered into the oesophagus which would increase the risk of aspiration to the lung.

The Applicants have identified the problems associated with the prior art and surprisingly discovered that a combination of being able to detect at least one stomach-related marker and carbon dioxide provides a much more reliable means for determining the location of, for example, a NG feeding tube in a subject.

The detection means for carbon dioxide and at least one detection means for a stomach-related marker may be disposed on at least one substrate.

The substrate may comprise a matrix. Advantageously, the substrate comprises a cellulose-based matrix. The substrate may be porous and/or perforated to permit the flow of fluid therethrough.

In an embodiment, the device comprises two or more detection means for a stomach-related marker. The two or more detection means may be disposed on the same substrate or different substrates.

The substrate may be an adapted form of pH filter paper, impregnated with a dye which changes colour from purple to yellow in the presence of carbon dioxide. Alternatively, the substrate may comprise adapted pH paper carrying sulfonephthalein or Bromothymol sulfonephthalein, which is an acid-base indicator.

The detection means for carbon dioxide may be capable of distinguishing the level of carbon dioxide present. A known carbon dioxide detector (available form Mercury Medical http://www.mercurymed.com/catalogs/ADR_CarbonDioxideDetector.pdf) changes colour depending upon the level of carbon dioxide present. 5% carbon dioxide detected in a sample is indicative of normal exhalation value and would indicate that the NG tube is located in the lung of a subject. Levels below that would indicate that the NG tube is either not in the lung or that the subject may be experiencing other medical problems, particularly where a stomach-related marker is not detected, indicating that the tube may possibly be in the lung.

The subject may be a mammal. Advantageously, the subject is a human.

### BRIEF DESCRIPTION OF THE FIGURES

Preferred embodiments of the invention shall now be described, by way of example only, with reference to the accompanying examples and drawings in which:

FIG. 1 is a perspective view of first embodiment of an in-line tester in accordance with the invention;

FIG. 2 is an exploded view of the in-line tester of FIG. 1;

FIG. 3 is a sectioned view of the in-line tester of FIG. 1;

FIG. 4 is a plan view of decals suitable for use with the in-line tester of FIG. 1;

FIG. 5 is a plan view of decals suitable for use with the in-line tester of FIG. 1;

FIG. 6 is a schematic plan view of a second embodiment of an in-line tester in accordance with the invention;

FIG. 7 is a schematic sectional view of FIG. 6 on VII-VII;

FIG. 8 is a schematic sectional view of FIG. 6 on VIII-VIII;

FIG. 9 is a schematic sectional view of FIG. 6 on IX-IX;

FIG. 10 is an exploded view of FIG. 9;

FIG. 11 is a schematic circuit diagram of the embodiment of the in-line tester of FIGS. 1 to 3;

FIG. 12 is a schematic circuit diagram of the embodiment of the in-line tester of FIGS. 6 to 9;

FIG. 13 is a schematic exterior view of an in-line tester as shown in FIG. 6;

FIG. 14 is a schematic circuit diagram showing a variation of the circuit of FIG. 12 incorporating a coarse liquid-stop device;

FIG. 15 is a circuit, in accordance with the invention;

FIG. 16 is a schematic cross-sectional view of an in-line tester in accordance with the invention;

FIG. 17 is a perspective view of an in-line tester in accordance with the invention;

FIG. 18 is an exploded view of the in-line tester of FIG. 16;

FIG. 19 is a computational fluid dynamic representation of liquid flow into the in-line tester of FIGS. 15 to 18;

FIG. 20 is a computational fluid dynamic representation of liquid flow into the in-line tester of FIGS. 15 to 18;

FIG. 21 is a computational fluid dynamic representation of liquid flow into the in-line tester of FIGS. 15 to 18;

### DETAILED DESCRIPTION

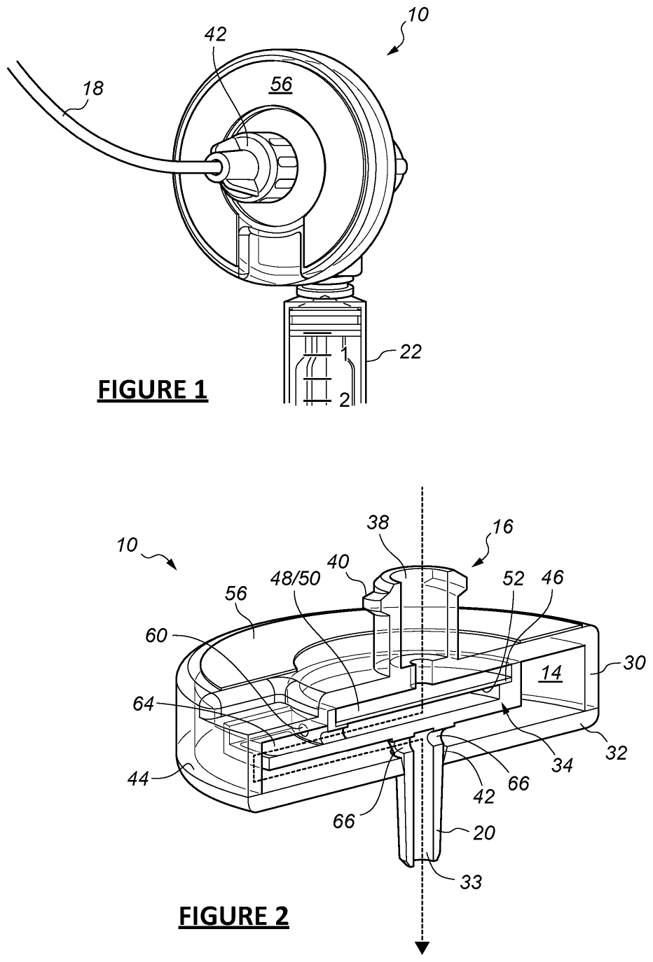

Referring to FIGS. 1 to 5 of the drawings, the in-line tester 10 comprises a main body 12 manufactured via a plastics injection moulding process from a transparent material, such as ABS. The main body 12 defines a hollow interior chamber 14 and has an inlet 16 connectable, in use, to an NG tube 18, and an outlet 20, connectable in use to a vacuum source (e.g. a vacuum pump; or a syringe 22—as shown in FIG. 1). A vacuum is applied to the outlet 20 of the in-line tester 10 to draw a sample of aspirate (gas and/or liquid) from within a patient via the NG tube 18, and the aspirate enters the hollow interior chamber 14 of the in-line tester 10 via the inlet 16.

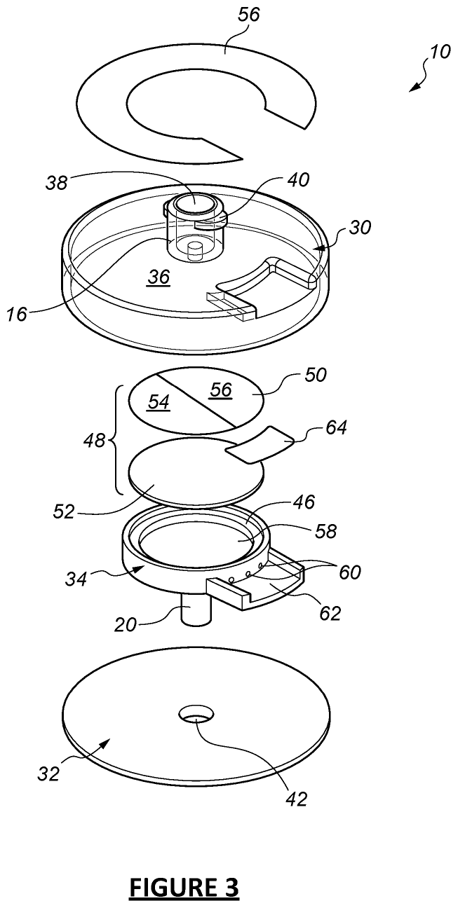

Referring to FIGS. 2 and 3 in particular, the main body 12 is formed by three main parts, namely: a generally dish-shaped first part 30; an annular back plate disc 32; and an insert 34.

The inlet 16 is formed as a tubular spigot extending concentrically from the outer face 36 of the generally dish-shaped first part 30. The inlet 16 has a through hole 38 that provides a fluid communication pathway into the interior of the main body 12, and also has external screw thread formations 40 for engaging complementary internal thread formations of a Luer-type connector 42 at the end of the NG tube 18.

The insert 34 is mostly located within the generally dish-shaped first part 30, but has an outlet spigot 20 formed integrally therewith, which sealingly extends through a tapered central through hole 42 in the annular back plate disc 32. The outlet spigot has a blind hole 33 in it (explained in greater detail below) and a plain outer surface, which can connect to the inlet of a syringe 22, or to a vacuum pump (not shown).

As can be seen by comparing FIGS. 2 and 3, the generally dish-shaped first part 30 has a planar peripheral edge 44, which is sealingly connected (for example by gluing or welding) to the outer periphery of the annular back plate disc 32. The insert 34 is thus retained in-situ.

The insert 34 has a generally circular dish-shaped profile, with an internally-rebated lip 46, which retains, by frictional engagement, a circular porous or perforated element 48 (first tester incorporating a liquid-stop device). The dimensions of the lip 46 are sized so as to form a valve seat against which the porous or perforated element sealingly seats. A seal can be perfected, if necessary, using a bead of sealant or adhesive (not shown).

As can be seen from the drawings, the porous or perforated element 48 comprises an air-permeable membrane, which permits air to pass through it, but not fluids. In the illustrated embodiment, the porous or perforated element 48 is both a pH tester and a liquid-stop device, and thus comprises two components, namely a liquid-absorbent layer 50, such as paper (located closest to the inlet 16), and a porous or perforated hydrophobic layer 52 (downstream of the liquid-absorbent layer 50). The two layers 50, 52 are conjoined to form a laminated structure, although they may equally be just clamped or otherwise held together. The main advantage of putting the two layers in close proximity, or touching one another, is that the wetting of the liquid-absorbent layer 50 very quickly, if not immediately, also wets the liquid-stop device, i.e. porous or perforated hydrophobic layer 52. Thus, the wetting of the first layer 50 causes the liquid-stop layer 52 to automatically close off almost immediately. However, in other embodiments, there may be a separation between these two layers 50, 52, or indeed, they may be located in entirely different regions of the in-line tester 10.

In this embodiment, the liquid-stop device, i.e. the hydrophobic layer 52, comprises pores or perforations that enable, when dry, air to pass through them (i.e. through the hydrophobic layer 52). However, when wetted by liquid, e.g. liquid absorbed by the liquid-absorbent layer 50, the liquid is repelled from the surface of the hydrophobic layer 52 and forced to overlie the less hydrophobic regions, that is to say, the openings of the pores or perforations. Provided the pores or perforations of the hydrophobic layer 52 are small enough (i.e. significantly smaller than the size of a liquid droplet), the liquid that overlies the pores or perforations effectively blocks the pores or perforations, thus inhibiting or preventing the passage of air or liquid through them. The aforesaid configuration conveniently converts the in-line tester 10 into a self-closing valve that permits air or gasses to pass through it when the porous or perforated element 48 is dry, but which when the porous or perforated element 48 is wetted, self-seals to prevent fluids from passing through it and further downstream.

The liquid-absorbent layer 50 is coated or impregnated, in the illustrated embodiment, in two discrete areas 54, 56, by different colorimetric substances adapted to exhibit a colour change in the presence of different target substances.

In one embodiment, the first area 54 is adapted to change colour according to the pH of an aspirated liquid, and the second area 56 is adapted to change colour in the presence of a target stomach-related marker, as described herein. The colour of the two regions 54, 56 can be viewed from without the tester 10 via the transparent generally dish-shaped first part 30. A generally C-shaped decal 56, comprising a colour chart corresponding to the or each colorimetric substances is affixed to the outer surface 36 of the generally dish-shaped first part 30. In the illustrated embodiment, the generally C-shaped decal 56 surrounds, and slightly overlaps, the porous or perforated element 48 so that a visual comparison of colour of the porous or perforated element 48 to the decal 56 can be made.

In one embodiment, the liquid-absorbent layer is manufactured of litmus paper, and this forms the first area 54, such that the acidity/alkalinity of the aspirate can be tested. However, the second area 56 is a stomach-related marker detector, which is coated, or impregnated, with tributyrin, which tests for gastric lipase. Tributyrin will produce alcohol and butyric acid on contact with gastric lipase, and the butyric acid will lower the pH on the litmus paper, giving an acidic pH reading. This method can effectively correct the “false negatives” of relatively high pH in gastric aspirates from patients on antacids.

Below (downstream of) the porous or perforated element 48, the insert 36 comprises an internal chamber 58 into which aspirated gasses are vented after having passed through the porous or perforated element 48. The side wall of the insert 34 comprises one or more through apertures 60, through which the aspirated gasses are vented, in use. The apertures are located slightly above a shelf part 62 of the insert 34, upon which is located a colorimetric capnometer 64 in the form of a paper test strip impregnated with a substance that undergoes a colour change in the presence of greater than 5% CO2. The shelf part 62 has a base 63 and two side walls 65, which serve to frictionally retain the colorimetric capnometer test strip 64 when the device is assembled. This particular configuration usefully causes the aspirated gasses to be concentrated, and to vent over the surface of, the colorimetric capnometer test strip 64, thus enabling the colorimetric capnometer 64 to test more effectively for the presence of relatively low concentrations of CO2 in the aspirated gas.

The colorimetric capnometer test strip 64 comprises a dye carrying substrate (such as used in Mercury Medical carbon dioxide detector http://www.mercurymed.com/catalogs/ADR_CarbonDioxideDetector.pdf), which functions by detecting acid formed in exhalation of carbon dioxide form a subject. Indicator colour is indicative of the following conditions: Blue—No CO2 is present; Green—Between 1%-2% CO2 is present; Yellow 5% or more CO2 is present.

The aspirated gas then flows out of the in-line tester 10, via the outlet 20, and this is accomplished by the provision of through holes 66 extending through the side wall of the outlet spigot 20 upstream of the annular back plate disc 32.

The flow path of aspirate is indicated schematically, in FIG. 3, by the chain-dash arrow 70, that is to say, in via the inlet 16, through the porous or perforated element, out through the apertures 60 and over the colorimetric capnometer 64, under the insert 34, through holes 66 and out through the outlet 33, 20.

In use, the inlet 16 is connected to an NG tube 18 and an aspirate is drawn up the NG tube 18 and into the device 10. Initially, gas containing carbon dioxide will flow form the patient into the device, passing through the porous pH indicator 260 substrate, through the bores 223 and contact the carbon dioxide detecting substrate 280. If the carbon dioxide level is above a predetermined threshold to indicate exhaled air, then a colour change occurs in the carbon dioxide detecting substrate which if positive is indicative of the NG tube being located in the lung of a patient.

Subsequent aspiration results in liquid entering the device which is absorbed by liquid absorbent layer. If stomach acid is present, the pH detector will change colour to indicate the presence of acid, and if stomach enzyme or another target stomach-related substance is present, then an acid will be catalyzed causing a pH indicator to show the presence of an acid.

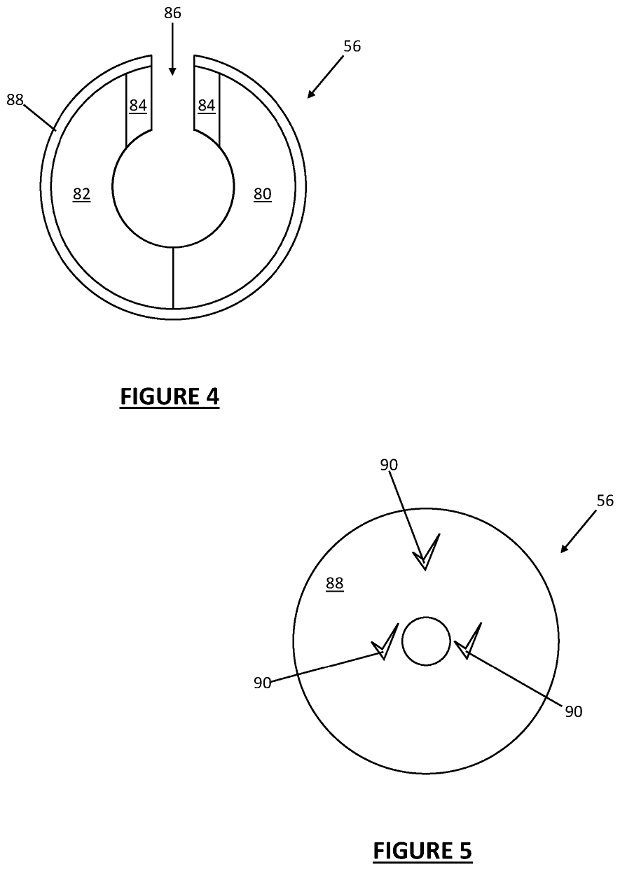

The decal 56 referred to previously, has different colour comparison areas, as shown in FIG. 4 of the drawings, against which the respective colorimetric test strips can be compared, in use, by a medical practitioner. In the example shown in FIG. 4, there are four areas. A first area 80 comprises a colour chart corresponding to the first area 54 of the porous or perforated element 48; a second area 82 comprises a colour chart corresponding to the second area 56 of the porous or perforated element 48; and a third area 84 (located either side of a cut-out 86) corresponding to the colorimetric capnometer. Thus, a practitioner can compare each of the three colorimetric test strips/areas against their corresponding colour charts. An outer peripheral region 88 is also provided, for displaying text, logos, instructions, CE markings etc.

In an alternative embodiment, as shown in FIG. 5, the decal 56 is adapted to cover most/all of the upper surface of the main body. In this embodiment, its colour corresponds to a “fail” colour for each of the tests (which may be a different colour in different areas of the decal 56). The decal 56 has tick-shaped through apertures cut into it, such that if a “positive” test result is confirmed using each of the thee colorimetric tests, the respective test trip/area will become visible, due to a difference in colour, compared with the regions of the decal 56 surrounding each cut out 90.

The aforedescribed embodiment of the invention has been found, in clinical trials, and tests, to provide a solution to one or more of the problems outlined in the introductory part of this disclosure, namely providing a double - or triple check for the correct positioning, or otherwise, of the tip of an aspirator tube in a patient. However, in certain situations, practice/protocols dictate other NG tube use methodologies. For example, in certain hospitals/environments, patients are intubated each time a feed is to be given. In this case, a clean, empty NG tube is inserted into the patient immediately prior to each feed, and after each feed, the NG tube is withdrawn and then discarded. In these situations, the NG tube is empty prior to aspiration of gases/liquids, in which case the aforedescribed embodiment of the invention has been shown to work satisfactorily.

In other hospitals/environments, however, the feed protocols can be somewhat different. By way of an example, in certain hospitals, where a patient is receiving ongoing nasogastric feeding, the NG tube is kept in place and is only withdrawn/replaced at certain intervals. As such, the same NG tube may be used to feed a patient several times before it is eventually discarded and replaced. In these situations, except for the first time the NG tube is used, the NG tube will inevitably contain some liquid—be that stomach liquid, residual feed from the previous feed, or a saline flush solution. As such, when the NG tube is used subsequently and an aspirate taken, the first few drops of aspirate may not be representative of the liquid surrounding the tip of the NG tube, but rather may be representative of the residual contents of the NG tube. When the previously-described embodiment of the invention is used, in these situations, it can result in the aspiration test procedure being stopped prematurely, for example when a drop of saline flush liquid, or residual feed from within the tube, is aspirated onto the pH test strip, which according to the afore described embodiment, would result in the in-line tester being “hydraulically locked” by the liquid-stop device becoming wet. As such, in these situations, the aforedescribed in-line tester may be somewhat ineffective because, by virtue of its self-closing feature, it is unable to test a sample of aspirate from the region surrounding the NG tube's tip, but rather simply tests an aspirate that was already in the NG tube prior to the test commencing. A need therefore exists for a further embodiment of the invention, which addresses this particular issue.

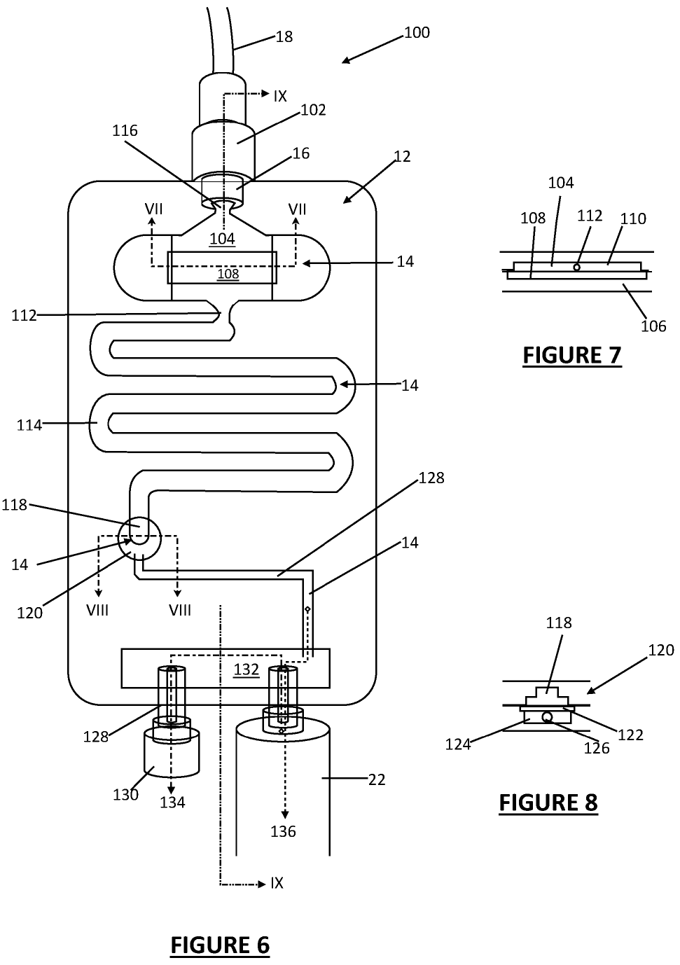

Referring now to FIGS. 6 to 10 of the drawings, another embodiment of an in-line tester 100 in accordance with the invention is shown. In this embodiment, the in-line tester 100 comprises a main body 12, again manufactured via a plastics injection moulding process, from a transparent material, such as ABS. The main body 12 defines a hollow interior chamber 14 as shall be described hereinbelow.

The in-line tester has an inlet 16, which is connectable, in use, to an NG tube 18 via a luer connector 102. The inlet 16 connects the NG tube 18, to a first portion 104 of the chamber 14. The first portion 104 of the chamber 14, which is shown in cross section in FIG. 7, comprises a base wall 106, which supports a small strip of pH indicator paper 108. A cavity 110 is provided on one side of the pH test strip 108, such that fluid entering the in-line tester 100, via the inlet 16 is able to come into contact with at least one surface or part thereof.

The first chamber part 104 has an outlet 112, which leads to a reservoir part 114 of the chamber 14.

The inlet 16 comprises a restriction, in the form of a Venturi 116 in a preferred embodiment, which causes liquid drawn up through the NG tube 18 to be “sprayed” over the exposed surface of the pH test strip 108. The provision of a constriction or Venturi 116 serves to cause the incoming liquid to “fan-out” as it enters the first chamber part 104, thereby ensuring that an adequate area of the pH test strip 108 is wetted by the incoming liquid.

The outlet 112 of the first chamber part 104 leads to a reservoir portion 114 of the chamber 14. In the illustrated embodiment, the reservoir 114 comprises a serpentine pathway within the main body 12, which can retain approximately 4 ml of liquid, when full. The volume of the reservoir 114 is ideally selected to be slightly larger than the internal volume of the NG tube 18 and so the exact volume of the reservoir is not fixed. The reason for providing a reservoir 114 is to enable a quantity of liquid within the NG tube 18 to be accumulated within the main body 12 of the in-line tester 110, for reasons that shall become apparent later.

The reservoir part 114 of the chamber 14 comprises an outlet 118, which feeds into a further test chamber part 120 of the chamber 14. The further test chamber part 120 is shown in cross section in FIG. 8 of the drawings, from which it can be seen that the outlet 118 of the reservoir is in fluid communication with a small disc tester 122 trapped, by its peripheries, between an upper part and a lower part of the main body 12. The test disc 122 is a CO2 tester, which exhibits a colour change in the presence of carbon dioxide.

The test disc 122 can be manufactured from a perforated, hydrophobic material, which enables gases exiting the reservoir 114 via its outlet 118 to pass through the test disc 122 and into a lower part 124 of the second test chamber 120, before exiting via an outlet 126 into a further pathway 128.

As gases are aspirated through the NG tube 18, they pass through the first chamber part 104, through the reservoir part 114 and eventually into the second test chamber 120, where they interact with the test disc 122. If the gas contains carbon dioxide, it will cause the test disc 122 to change colour, which will be visible to a user (not shown) observing the in-line tester 110, through its transparent main body portion. The aspirated gas can be drawn into a connected vacuum source, in the illustrated example, a syringe 22, and the aspirated gas can, therefore, be tested for the presence or otherwise of carbon dioxide.

In other embodiments of the invention, the test disc 122 is formed from two parts, namely a downstream colorimetric test disc 122, and an upstream porous or perforated element comprising a hydrophobic material 52. In this embodiment of the invention, the two parts work together as previously described, namely with the colorimetric test disc 122 being able to test for the presence of CO2, with the porous or perforated, hydrophobic layer 52, acting as a liquid-stop device 52 upstream of the colorimetric capnometer. Again, the two layers 122, 52 need not necessarily be in intimate contact with each other, although this may be beneficial in certain circumstances. Nevertheless, there is a liquid-stop device 52 located upstream of the CO2 tester, which means that when the test is complete, that is to say when a sample of aspirated liquid comes into contact with the porous or perforated, hydrophobic layer 52, the in-line tester 100 is essentially hydraulically locked, thereby preventing further aspiration. Meanwhile, a sample of aspirated gas will be trapped downstream of the liquid-stop device 52, thereby inhibiting or slowing the reversion of any colour change in the colorimetric capnometer for a certain period of time.

It is possible to connect the outlet of the in-line tester to a vacuum pump, or, in a preferred embodiment to a syringe 22.

Where a small syringe 22 is used, it may be necessary to repeatedly withdraw the syringe plunger (not shown) to obtain a sufficient test volume via the NG tube 18. To accomplish this, the in-line tester 110 is provided with a secondary outlet 128, to which is connected a one-way valve 130. The syringe 22 and one-way valve 130 are operatively interconnected via a chamber 132 formed in the main body 12 of the in-line tester 100. The one-way valve 130 has a particular “cracking pressure”, above which, the valve will open to allow gas to be expelled 134 from the in-line tester. The cracking pressure of the one-way valve 130 is designed to be lower than the permeability of the liquid-stop device such that upon depression of the syringe plunger (not shown) fluid is forced from the syringe 22, via the chamber 132 and out via the one-way check valve 130, as indicated by arrow 134 in FIG. 6. However, upon withdrawing the plunger, the one-way check valve 130 closes, thus enabling fluid to be drawn up through the NG tube, through the in-line tester 100, and, ultimately, into the syringe, as indicated by arrow 136.

The benefits of this particular configuration of the invention are manifold.

In particular, because the main body 12 of the in-line tester 100 is manufactured from a transparent plastics material, it is possible for a user (now shown) to observe the progression of aspirated fluids through the tester 100. This usefully enables a user of the device to “see” when e.g. saline flush has been aspirated, followed by stomach content, for example, by a colour change in the reservoir.

In a first example, where the NG tube 18 is initially empty, upon repeated withdrawal and compression of the syringe 22 plunger, fluid will be “pumped” up through the NG tube in the manner previously described. Because the NG tube 18 is initially empty, the first fluid that will be drawn into the in-line tester 100 will be gas/air from within the NG tube itself or the patient's stomach/lung. This gas/air will simply flow through the first chamber part and over the pH test strip 108, through the reservoir 114, where it will eventually come into contact, and pass through the CO2 test disc 122.

The user, by observing the colour of the CO2 test disc 122 will be able to ascertain whether it is an air/stomach gas sample, or whether it is a “breath” sample of air aspirated from the patient's lung, for example.

Eventually, liquid (hopefully gastric juice) will be aspirated up the NG tube 18, where it will enter the in-line tester 100 via the inlet 16. The aspirated liquid will be “sprayed” by the Venturi 116 at the tester inlet 116, and the design of the Venturi 116 is such that aspirated liquid is sprayed/dispensed over the exposed surface of the pH test strip 108. By observing a colour change in the pH test strip 108, a user (not shown) will be able to determine whether an acidic, (eg a gastric juice) sample has been aspirated, or whether something else has been aspirated.

Liquid will then be drawn into the reservoir 114, where it will gradually fill the reservoir and the user will be able to observe the progress of the liquid as it is drawn into the reservoir.

In a second situation, for example where the NG tube 18 has been flushed with water or saline prior to use, it will initially contain a quantity, typically 4 ml, of saline solution or water. In this situation, the “first liquid” to come into contact with the pH test strip 108 ought to be neutral, which might, ordinarily, indicate incorrect placement of the NG tubes tip in the patient's stomach. However, this could be a simple “false negative” because the first liquid aspirated is, in fact, saline solution rather than gastric juice. The test therefore needs to continue until such time as the flush liquid within the NG tube 18 has been recovered, which will, hopefully, be followed by a sample of gastric juice.

Therefore, the invention comprises a reservoir 114, into which this initial liquid may be accumulated. Here, the volume of the reservoir 114 is slightly greater than the internal volume of the NG tube 18 such that when the reservoir is full, the user knows that what is being tested ought to be gastric juice, rather than flush liquid. The user (not shown) is therefore able to observe the progression of the flush liquid through the system, by observing the reservoir, which is visible from outside the in-line tester 100 by virtue of it being manufactured from a transparent plastic, and, ultimately, to test the pH of a gastric juice sample thereafter.

Although not shown in FIG. 6 for clarity, the in-line tester 100 suitably comprises one or more decals, each comprising a colour chart corresponding to the or each colorimetric substances. The decal or decals are suitably affixed to an outer surface of the in-line tester 100, adjacent to, but preferably slightly overlapping the testers 108, 122—so that a visual comparison of colour of the testers 108, 122 to the colours or other indications on the decal can be made.

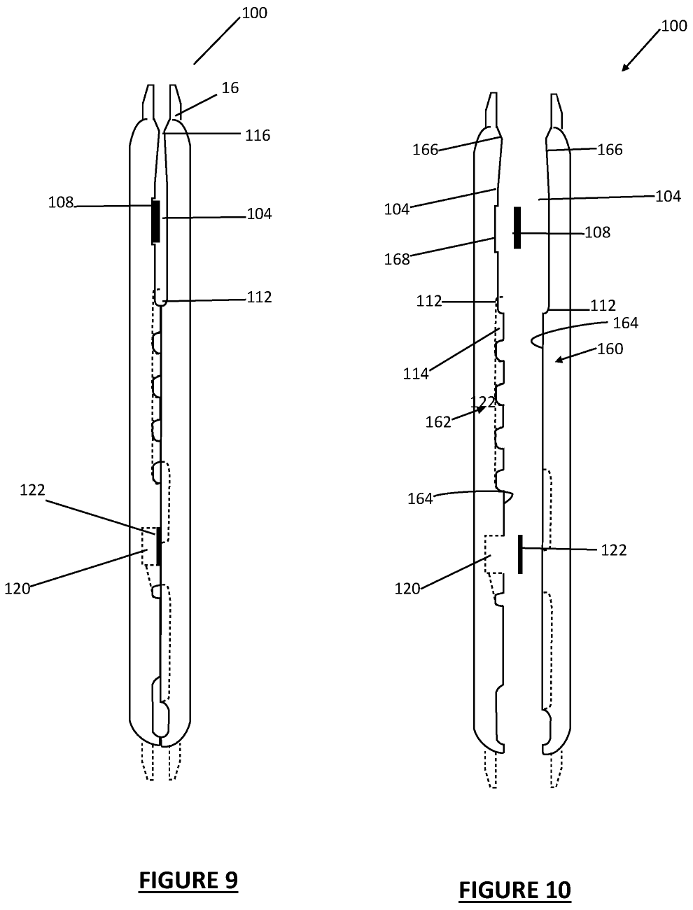

The in-line tester 100, shown in FIGS. 6, 7 and 8 of the drawings, is shown, schematically, in cross section in FIG. 9, which is a cross section of FIG. 6 on IX-IX.

In a preferred embodiment of the invention, the main body 12 of the in-line tester 100 is manufactured from two plastics injection moulded components, which fit together to form the device shown, schematically, in FIG. 6. The various chambers 104, 120, 132 can be formed simply by providing recesses or grooves in the mating surfaces of the two components.

Referring to FIGS. 9 and 10 of the drawings—FIG. 10 being an exploded view of FIG. 9 the inlet 16 is formed in two halves from each of the main body pieces 160, 162. The two parts 160, 162 have a flat mating surface 164, which when pushed together, form a fluid-tight seal between the two pieces 160, 162. Channels or cavities within the in-line tester 100 can be formed by providing recesses or cavities in each of those mating surfaces 164.

Referring to FIGS. 9 and 10, it can be seen that the Venturi 116 is formed by a pair of opposing inclined surfaces 166 formed in each of the main body pieces 160, 162. The first cavity 104, which houses the pH test strip 108 is likewise formed with an additional recess part 168 formed in one of the pieces 162, for locating and retaining the pH test strip 108.

The outlet 112 of the first chamber part 104 is formed by complimentary recesses formed in each of the pieces 160, 162.

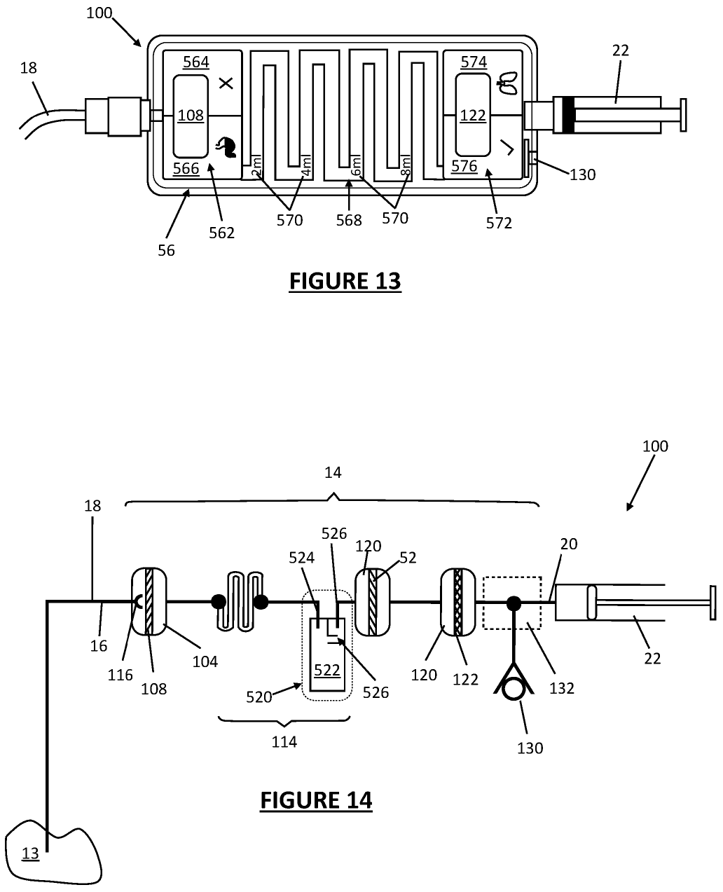

By way of example, an in-line tester 100 similar to that described above with reference to FIGS. 6 to 10 of the drawings, is shown in FIG. 13, in which a partially transparent decal 56 covers a front face of the in-line tester 100. The decal 56 has a first part 562, which surrounds the pH test strip 108 viewing window. The first part 562 is divided into two differently-coloured regions 564, 566, which are coloured to match the colour of the pH paper 108 when a “fail”/“do not feed”; or a “pass” condition is detected, respectively.

The reservoir 114 part of the in-line tester 100 is visible through a transparent 568 part of the decal 56. Graduations 570, indicating the volume of aspirated liquid are optionally provided.

A third part 572 of the decal 56, which surrounds the CO2 test strip 122 viewing window. The third part 572 is divided into two differently-coloured regions 574, 576, which are coloured to match the colour of the CO2 paper 122 when a “fail”/“do not feed”; or a “pass” condition is detected respectively. The two regions 574, 576 are indicated for the avoidance of doubt by “tick” and “lung” indicia respectively.

The serpentine reservoir 114 is formed by a correspondingly shaped serpentine groove formed in one of the main body pieces 162.

Likewise, the second chamber 120 is formed by a relatively deeper depression in the second main body part 162 and that enables the co2 test disc 122 to be housed therein. Further recesses formed in the main body pieces 160, 162 form the various other channels/cavities as indicated, schematically in FIGS. 9 and 10.

The two main body pieces 160, 162 can either be glued together, for example, by using an adhesive or welding along the shut line to form fluid-tight cavities/channels within the main body 12, or, where the mating surfaces 164 are sufficiently flat and/or intimate, such sealing may be accomplished by simply clamping, clipping or otherwise holding together, the two main body pieces 160, 162.

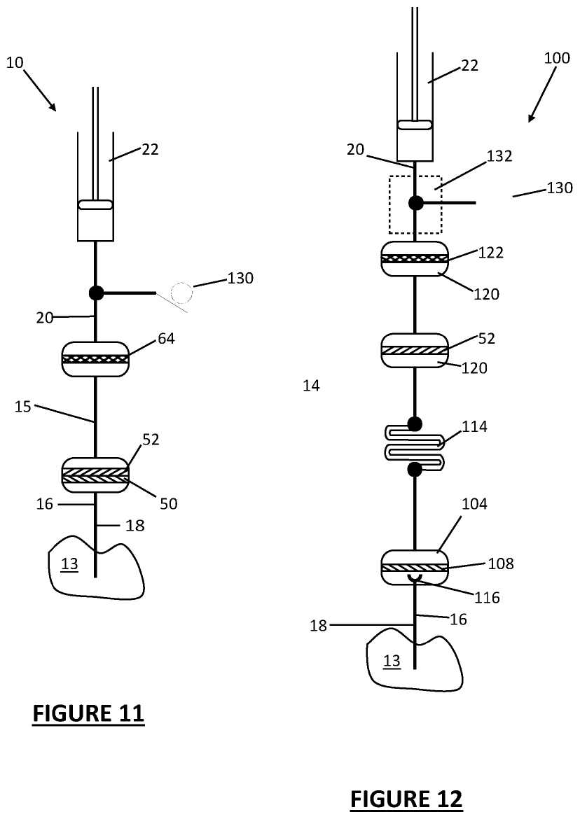

Referring now to FIGS. 11 and 12 of the drawings, which are schematic circuit diagrams for the in-line testers 10, 100 shown in FIGS. 1 to 3 and 6 to 9 above, respectively.

In FIG. 11, the in-line tester 10 is fitted to an NG tube 18 at its inlet 16, and to a syringe 22 at its outlet 20. The tip of the NG tube is placed in the stomach 13 of a patient (not shown).

The in-line tester 10 comprises a chamber 14, which houses a first tester, namely a disc of pH test paper (e.g. litmus paper) 50, which is backed by a liquid-stop device, namely a perforated plastics, hydrophobic disc 52, which when wetted by aspirated liquids, closes-off and stops/inhibits further aspiration. A fluid passageway 15 connects the chamber 14 to a further chamber that houses a colorimetric capnometer 64, in this case, a strip of CO2-sensitive indicator paper that changes colour in the presence of CO2.

The syringe's plunger can be withdrawn to aspirate a sample of fluid from the stomach, via the NG tube 18 and into the in-line tester 10.

Aspirated gas passes through the pH test paper 50 and the liquid-stop device 52, where it then comes into contact with the colorimetric capnometer 64 to indicate the presence or otherwise of CO2 in the aspirated gas sample.

Thereafter, liquids may be aspirated from the stomach 13, via the NG tube 18, where they come into contact with the first tester 50 and indicate the presence, or not, of a target substance, e.g. stomach acid and/or a substance (e.g. a protein) only found in the stomach 13. The aspirated liquid contacts the liquid-stop device 52, causing the in-line tester 10 to hydraulically lock, thereby signifying the end of the procedure.

A one-way valve 130 may optionally be provided in a branch spurred-off between the syringe 22 and the outlet 20. This enables the syringe plunger to be depressed, and fluid within the syringe 22 to be vented via the one-way valve 132. This configuration permits a relatively small syringe 22 to be used as part of a pump, rather than having to use a relatively large syringe to obtain an adequate quantity of aspirate.

Referring now to FIG. 12, the in-line tester 100 is fitted to an NG tube 18 at its inlet 16, and to a syringe 22 at its outlet 20. The tip of the NG tube is placed in the stomach 13 of a patient (not shown). The in-line tester 100 comprises a chamber 14, which has several parts.

A first chamber part 104 houses a first tester, for example a strip of pH test paper (e.g. litmus paper) 108 and/or a strip of other indicator paper, which changes colour in the presence of a target substance.

The first chamber part 104 is connected to a reservoir 114, which can accumulate a quantity of aspirated liquid.

The reservoir 114 connects to a further chamber 120, which houses a liquid-stop device 52, namely a perforated plastics, hydrophobic disc, which when wetted by aspirated liquids, closes-off and stops/inhibits further aspiration.

Downstream of the liquid-stop device 52, there is provided a colorimetric capnometer 122, in this case, a disc of CO2-sensitive indicator paper that changes colour in the presence of CO2.

A one-way valve 130 is provided in a branch spurred-off, e.g. via a chamber 132, between the syringe 22 and the outlet 20.

The syringe's plunger can be withdrawn to aspirate a sample of fluid from the stomach, via the NG tube 18 and into the in-line tester 10. Aspirated gas passes through the first tester 108 and the liquid-stop device 52, where it then comes into contact with the colorimetric capnometer 122 to indicate the presence or otherwise of CO2 in the aspirated gas sample.

Thereafter, liquids may be aspirated from the stomach 13, via the NG tube 18, where they come into contact with the first tester 104 and indicate the presence, or not, of a target substance, e.g. stomach acid and/or a substance (e.g. a protein) only found in the stomach 13. The aspirated liquid then fills the reservoir 114 until it eventually contacts the liquid-stop device 52, causing the in-line tester 10 to hydraulically lock, thereby signifying the end of the procedure.

By virtue of the one-way valve 130, the syringe plunger to be depressed, and fluid within the syringe 22 can be vented via the chamber 132 and the one-way valve 132. This configuration permits a relatively small syringe 22 to be used as part of a pump, rather than having to use a relatively large syringe to obtain an adequate quantity of aspirate.

Referring now to FIG. 14 of the drawings, a slight variation of the circuit diagram shown in FIG. 12 is described. Identical reference signs have been used to identify identical features, for the avoidance of repetition, and for clarity. In FIG. 14, it can be seen that the in-line tester 100 has been modified by the addition of a coarse liquid-stop device 520 located upstream of the previously-described porous or perforated, hydrophobic membrane/disc 52. The coarse liquid-stop device 520 comprises a chamber 522, having an inlet 524 connected to the outlet of the reservoir 114, and an outlet 526 connected to the inlet of the chamber 120. Baffling 526 is provided within the chamber 522 to prevent liquid drops from being inadvertently splashed onto, or reaching the outlet 526. Here, liquids and gasses can be drawn into the coarse liquid-stop device 520, as may happen when the reservoir 114 is full, and the coarse liquid-stop device 520, provides a further means for preventing the porous or perforated, hydrophobic membrane/disc 52 from wetting out, as liquid droplets will be collected in the chamber 522, rather than passing through the coarse liquid-stop device 520 to the porous or perforated, hydrophobic membrane/disc 52 downstream of it.

The main purpose of the coarse liquid-stop device 520 is that it enables, where the reservoir 114 is full of aspirated liquid, say saline flush, to nevertheless permit the passage of air/gas bubbles to the CO2 test strip 122, via the porous or perforated, hydrophobic membrane/disc 52. This may occur where an NG tube has been used previously and thus contains a saline flush liquid. However, if, somehow, the NG tube has become misplaced, as may happen where the patient “wretches” the NG tube back up their oesophagus, then the next time the NG tube needs to be used, it will be checked for correct placement. Now, the first few ml of aspirate will be saline flush, or residual feed within the NG tube, and this liquid will fill, or partially fill the reservoir 114. Subsequent aspiration eventually empties the NG tube 18, such that gas is now, finally aspirated. This aspirated gas will bubble through the liquid already in the reservoir, and without a coarse liquid-stop device 520 present, the liquid would tend to be splashed onto the porous or perforated, hydrophobic membrane/disc 52, thereby wetting it, and causing the in-line tester 100 to hydraulically lock. However, by placing a coarse liquid-stop device 520 upstream of the porous or perforated, hydrophobic membrane/disc 52, the aspirated gas is able to pass through the reservoir, and the porous or perforated, hydrophobic membrane/disc 52 before it reaches the CO2 paper 122, with the splashed liquid effectively being filtered-out by the coarse liquid-stop device 520. This enables a user to reliably test an aspirated gas sample—even after already having aspirated a liquid sample.

The coarse liquid-stop device 520, where provided, can be incorporated into the reservoir 114, or into the chamber 120, as desired.

### Example 1

Samples were used to generate the following truth table comparing a device in accordance with the present invention having means for pH detection, enzyme detection and carbon dioxide detection with the current clinical standard (pH paper).

There were three scenarios: 1) Normal pH content in stomach; 2) Patient on antacid medication; and 3) Stomach content in the lung. Each method and device was used and the results set out below.

TABLE 1

| | DEVICE IN | |
|---|---|---|
| | ACCORDANCE | |
| | WITH PRESENT | CURRENT CLINICAL |
| SCENARIO | INVENTION | STANDARD (PH PAPER) |
| Normal pH content | Confirmed | Confirmed |
| in stomach | | |
| Patient on antacid | Confirmed | False negative confirmation |
| medication | | tube not in stomach, patient |
| | | has delayed feed |
| Stomach content in | Confirmed | False negative confirmation |
| the lung | | tube not in stomach, patient |
| | | has delayed feed |

### Example 2

Samples were used to generate the following truth table comparing a device in accordance with the present invention having means for pH detection, enzyme detection and carbon dioxide detection with the current clinical standard (pH paper). In this example, the three markers tested for in the device according to the present invention are separated out to give a clearer demonstration of the advantages of a device in accordance with the present invention.

There were three scenarios: 1) Normal pH content in stomach; 2) Patient on antacid medication; and 3) Stomach content in the lung. The presence or absence of three markers was known and each method and device was used and the results set out below.

TABLE 2

| | 1^{ST} | 2^{ND} | 3^{RD} | DEVICE ACCORDING | CURRENT |
|---|---|---|---|---|---|
| | MARKER | MARKER | MARKER | TO PRESENT | CLINICAL |
| SCENARIO | PH | ENZYME | CO₂ | INVENTION | STANDARD (PH) |
| Normal pH content | Positive | Positive | Negative | 1^{st}, 2^{nd} and 3^{rd} | 1^{st} marker |
| in stomach | | | | markers confirmed | confirmed only |
| Patient on antacid | Negative | Positive | Negative | 1st, 2nd and 3rd | None or |
| medication | | | | markers confirmed | confirmed |
| Stomach content in | Positive | Positive | Positive | 1st, 2nd and 3rd | 1^{st} confirmed |
| the lung (or M. | | | | markers confirmed | only - dangerous |
| Cattarhalis infection | | | | | |
| in lung) | | | | | |

In complicated situations where for example, a patient is taking antacids or there is stomach content in the lung, only devices in accordance with the present invention will confirm the actual location of the NG tube for enteral feeding. Known devices and methods will only give an accurate indication of location when a patient has a normal stomach pH. In all other scenarios the result from using known devices can be dangerous and has potentially fatal consequences should feeding via the tube be initiated where an incorrect placement of the tube is mistakenly indicated as being in the stomach.

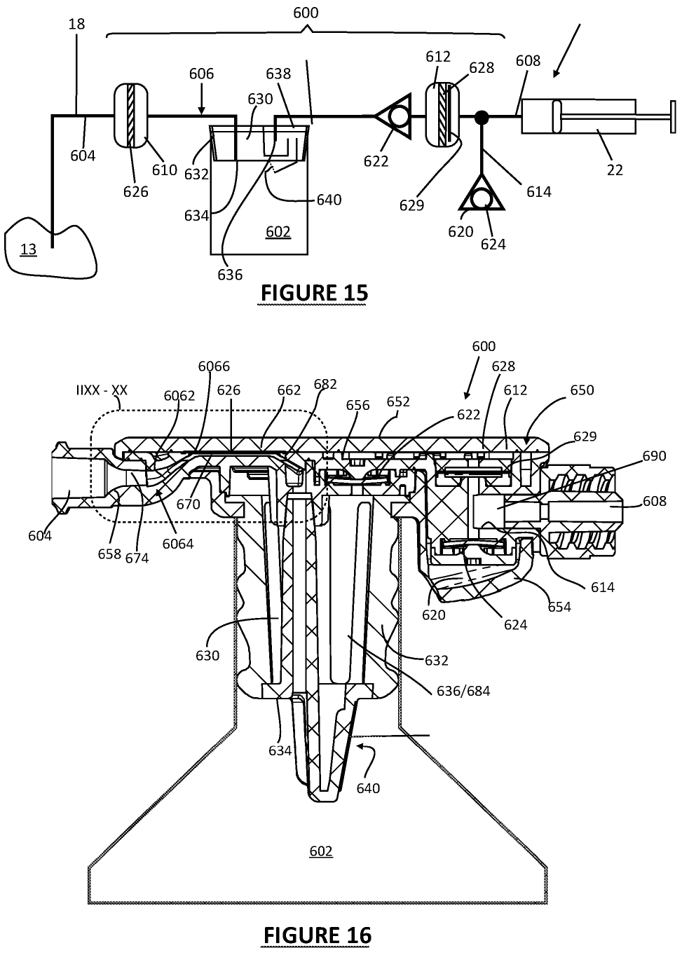

Referring now to FIGS. 15 to 21 of the drawings, an embodiment of an in-line tester in accordance with the invention is shown as a schematic circuit diagram, in cross-section, perspective and exploded views, respectively.

Referring firstly to FIG. 15 of the drawings, the in-line tester 600 of the invention differs from the embodiments described previously by virtue of its removable reservoir 602. The main body 600 has an inlet 604 that connects, in use, to an NG tube 18 whose tip is located in the stomach 13 of a patient. A fluid passageway 606 connects the inlet 604 to an outlet 608, which is connected, in use, to a vacuum source, such as a syringe 22. In-line with the fluid passageway 606 are first 610 and second 612 chambers and the reservoir 602, which is formed as a removable container. The fluid passageway 606 bifurcates into a limb 614 near to the outlet 608 and the limb 614 terminates in a vent port 620, which is open to atmosphere.

One-way valves 622, 624 are provided in the main fluid passageway 606 and the limb 614, respectively, and these valves 622, 624 ensure that when the syringe 22 plunger is withdrawn, fluids are drawn through the in-line tester 600 from inlet 604 to outlet 608, but when the syringe 22 plunger is depressed, air is vented through the vent port 620.

The first chamber 610 contains a pH-sensitive test strip 626, such as litmus paper or indicator paper. When fluids are aspirated up the NG tube 18 and into the in-line tester 600, they come into contact with the pH-sensitive test strip 626, which changes colour according to the acidity or alkalinity of the aspirated fluid.

Likewise, the second chamber 612 contains a CO2-sensitive test strip 628, such that when fluids are aspirated up the NG tube 18 and into the in-line tester 600, they come into contact with the colorimetric capnometer 628, which changes colour according to the concentration of CO2 in the aspirated fluid.

The fluid passageway 606 is interrupted by an attachment formation 630, which is formed as a truncated cone, whose outer surface comprises an elastomeric layer 632 that sealingly and detachably grips an inner surface of the reservoir container 602. The attachment formation 630 has two through holes forming ports 634, 636 that connect, respectively, to the fluid passageway 606 upstream of the reservoir container 602 and downstream of the reservoir container 602. The reservoir container 602 can be removed from the attachment formation 630 by pulling and/or twisting.

In order to prevent the CO2-sensitive test strip 628 from being wetted by liquids, a liquid-stop device or devices are provided. One liquid-stop device comprises a labyrinthine passageway 638 located upstream of the port 636. Another liquid-stop device comprises a porous or perforated element 640 (such as a web of perforated polymer and/or a web of paper or cardboard) overlying the port 636, whose pores are sized so as to permit the passage of gasses therethrough, but which inhibit or prevent the passage of liquids therethrough. A secondary liquid-stop device 629, in the form of a porous or perforated disc as previously described is interposed between the colorimetric and the capnometer 628 and the outlet 690. The secondary liquid-stop device 629 prevents liquids from being introduced into the in-line tester and coming into contact with the colorimetric capnometer 628 via the outlet 608. It can therefore be seen, from FIG. 15 of the drawings, the operational principles of the invention.

An actual embodiment of the invention is shown, schematically, in FIGS. 16, 17 and 18 of the drawings, in which the in-line tester 600 comprises a main body 650, which is formed from several snap-together pieces, as can be seen most clearly in FIG. 18 of the drawings. The main body 650 comprises a transparent, upper cap 652, which snaps onto a lower housing 654 sandwiching two additional components 656 and 658 therebetween.

The upper of the intermediate components 656 has an upper surface, which is provided with an aperture 662 into which a strip of pH-sensitive paper 626 is retained. The pH-sensitive paper 626 is retained laterally by the periphery 668 of the aperture 662, from below by a pad 670 of the lower internal component 658 and from above by the underside of the transparent upper cap 652.

A luer connector 672 is provided for connection to an NG tube 18 as previously described. The Luer connector 672 forms the inlet of the device and this communicates with a first part 674 of the fluid passageway 606, which feeds into the first cavity 610 defined by the aperture 662 containing the pH test strip 626. Thus, as fluids are drawn into the tester 600, they first come into contact with the underside of the pH indicator paper 626 and the colour of the pH indicator paper 626 can be seen through the transparent upper cap 652.

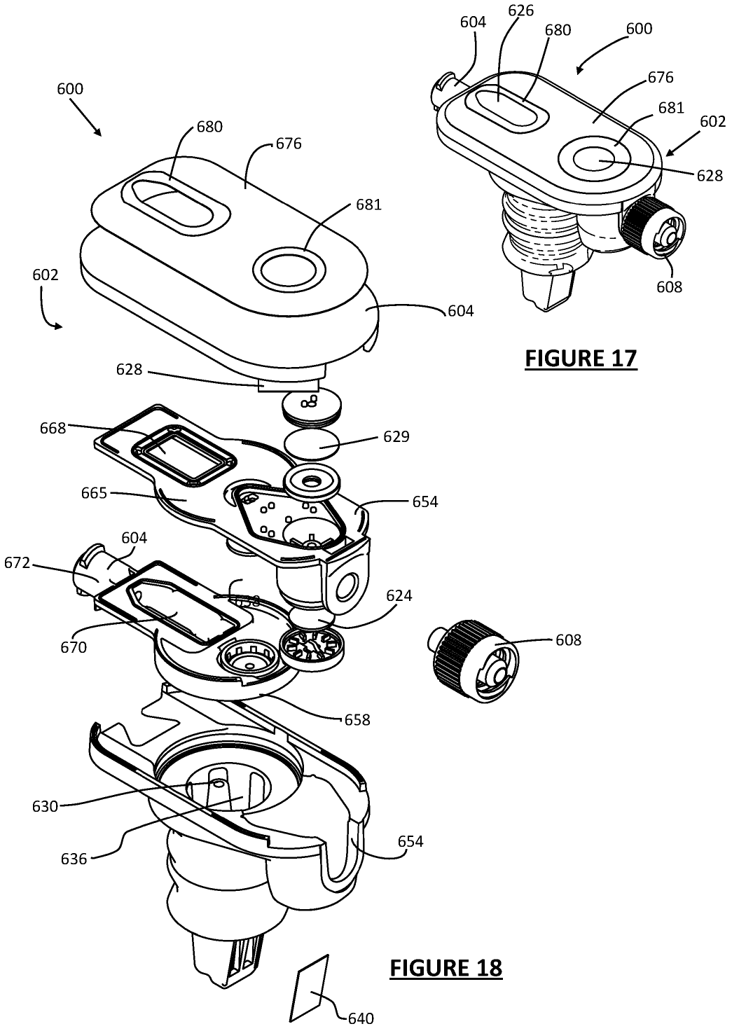

As can be seen in FIG. 17 and FIG. 18, a decal 676 is placed on top of the transparent upper cap 652, which has colour-coding charts 680 surrounding the location of the pH test strip 626 so that the colour of the pH test strip 626 can be compared against the colour chart 680 to determine the acidity/alkalinity of the aspirated fluid.

A second internal conduit 682 feeds from the downstream side of the first cavity 610 and into a reservoir 602. The reservoir 602 is formed as a bottle-type device, which push fits onto an elastomeric component 632 over-moulded onto a connection formation 630 extending from the lower cap 654, as can be seen clearly in FIG. 18 of the drawings. The elastomeric “bung” 632 provides a resilient seal against the internal wall of the reservoir 602 thereby enabling the reservoir 602 to be sealingly and detachably affixed to the main body 600 of the tester.

Fluid aspirated from the patient therefore flows through the second conduit 682 into the reservoir 602. The liquid component of the fluid collects in the reservoir 602, whereas the gas component of the fluid is able to flow up through a third conduit part 684 of the fluid passageway 606 as shall be described hereinbelow.

A liquid-stop device 640 is provided at the inlet side of the third conduit 684, and this prevents liquid from being drawn beyond the reservoir 602. Thus, although liquid and gas may be aspirated into the reservoir 602, only gas can be aspirated into the third conduit 684 and thence into the second chamber 612.

A one-way valve 622 is provided in the third conduit 684 before the fluid passageway 606 leads to the second chamber 612, which contains a colorimetric capnometer test disc 628. The colorimetric capnometer 628 changes colour in the presence of carbon dioxide, and the colour of that disc 628 can be viewed through the transparent upper cap 652 of the main body. Again, a colour chart 681 is provided on the decal 676 to aid interpretation of the colour of the disc 628.

A fourth internal conduit part 690 of the fluid passageway 606 connects the downstream side of the second chamber 612 to the outlet 608, which can be connected to a syringe or a vacuum pump. A further one-way valve 624 is provided and this is arranged to allow air to be expelled from the fourth conduit 690 and out through a vent aperture 620. The function of the one-way valves 622, 624 is to enable a vacuum to be applied by withdrawing a syringe plunger attached to the outlet 608, thereby enabling a vacuum to be drawn at the inlet 604, but also permitting air from the syringe 22 to be vented through the vent opening 620 upon depression of the syringe plunger.

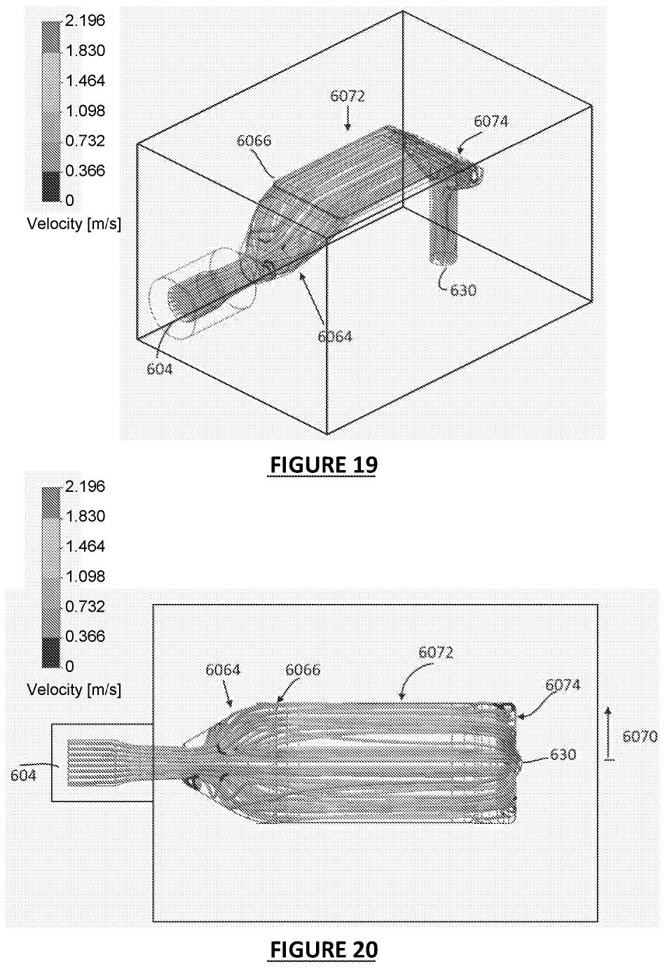

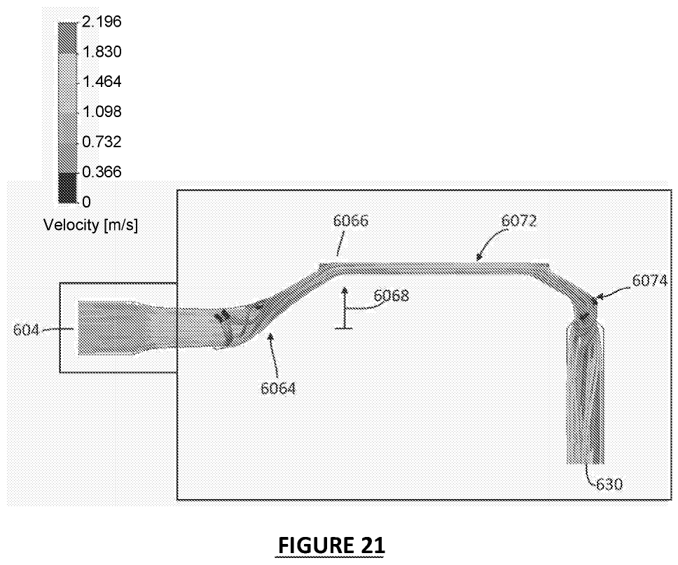

Finally, FIGS. 19, 20 and 21 of the drawings show CFD representations of liquid entering the in-line tester previously described with reference to FIGS. 15 to 18. The CFD analysis shows the configuration of the fluid passageway 606 between the inlet 604 and the entry point 630 into the reservoir 602 in perspective, plan and side elevation, respectively. Referring back to FIG. 16 of the drawings, the fluid passageway 6062 between the inlet 604 and the first chamber containing the colorimetric tester 626 has a generally circular cross-section at its entry point, which transitions into a substantially rectangular cross-section 6066, where it enters the first chamber. The transition is formed by way of a loft 6064, that is to say, a smooth transition between the circular inlet profile and the rectangular outlet profile 6066. It can be seen, particular from FIGS. 16 and 21 that the loft 6064 has a vertical component 6068 thus forming a “swan neck”, which serves to distribute the flow of liquid evenly. The loft 6064, as can be seen most clearly from FIG. 20 also has a lateral component 6070, which serves to spread the liquid laterally so as to form the substantially linear flow region 6072 whereby the liquid forms a “sheet” of liquid that progresses with a linear front across the rear surface of the colorimetric tester 626. A further downstream loft 6074 is also provided so as to transition the sheet of liquid in the linear flow region 6072 back into a substantially circular cross-section where it feeds into the reservoir 630.

The configuration of the fluid passageway 606 between the inlet 604 and the reservoir 602 is important to ensure that the entire surface of the colorimetric tester is evenly wetted using the lease possible amount of liquid. In a preferred embodiment of the invention, the volume of the fluid passageway between the inlet 604 and the secondary loft 6074 is less than 0.25 ml.

The invention is not restricted to the details of the foregoing embodiments, which are merely exemplary of the invention. For example, any shapes, sizes, relative dimensions etc. are illustrative, and not limiting, as are any material selections and/or design choices (e.g. type of check valve).

1-23. (canceled)

24. An in-line tester comprising main body, an inlet connectable in use to an NG tube, an outlet, connectable in use to a vacuum source, an exhaust port in fluid communication with air surrounding the main body and a fluid passageway interconnecting the inlet and the outlet,
the in-line tester additionally comprising, and each of the following being connected in-line with the fluid passageway:
a lofted fluid passageway comprising a loft that transitions from a circular cross-section to a rectangular cross-section, said lofted fluid passageway being located between the inlet and a first chamber containing a colorimetric tester, the rectangular cross-section being substantially perpendicular to an edge of the colorimetric tester of the first chamber such that, during aspiration, fluids entering the in-line tester via the inlet are directed so as to form a flat sheet of fluid with a substantially linear front that passes across a rear surface of the said colorimetric tester;
a second chamber also containing a colorimetric tester;
a first one-way valve in-line with the fluid passageway permitting the flow of fluids from the inlet to the outlet;
a liquid-stop device interposed between the first and second chambers that permits the flow of gasses, but which inhibits or prevents the flow of liquids, towards the second chamber; and
a reservoir interposed between the first and second chambers;
the in-line tester further comprising:
a second one-way valve permitting the flow of fluids from the outlet to the exhaust port; and the in-line tester being characterised by:
the reservoir comprising a container that is detachably connected to the main body; and
the main body comprising an attachment formation comprising:
a seal that sealingly and detachably connects the container to the main body,
a first port connecting the fluid passageway upstream of the attachment formation to the interior of the container; and
a second port connecting the fluid passageway downstream of the attachment formation to the interior of the container.

25. The in-line tester of claim 24, wherein the loft comprises an offset in a direction perpendicular to an axis of the inlet.

26. The in-line tester of claim 24, further comprising an additional liquid-stop device interposed between the second colorimetric tester and the outlet arranged so as to prevent or inhibit liquids introduced at the outlet from coming into contact with the colorimetric tester of the second chamber.

27. The in-line tester of claim 24, wherein the attachment formation comprises an attachment body extending from the main body, the first and second ports being formed as through holes in the attachment body.

28. The in-line tester of claim 24, wherein the seal comprises an elastomeric member over-moulded onto the attachment formation.

29. The in-line tester of claim 28, wherein the elastomeric member comprises a ribbed outer surface profile, the ribs forming one or more compressible sealing rings that friction-fit and seal to a complementary inner surface of the container.

30. The in-line tester of claim 27, wherein the liquid-stop device is located on the attachment body and sealingly covers the second port.

31. The in-line tester of claim 24, wherein the liquid-stop device comprises any one or more of: a labyrinthine liquid trap; a porous or perforated element whose pores are sized so as to permit the passage of gasses therethrough, but which inhibit or prevent the passage of liquids therethrough; and a web of perforated polymer and/or a web of paper or cardboard.

32. The in-line tester of claim 24, wherein the container is manufactured of a transparent material.

33. The in-line tester of claim 24, wherein the outlet is connectable, in use, to a syringe.

34. The in-line tester of claim 24, wherein the inlet and/or outlet comprise any one of: a luer; a luer-lock; a screw thread; a bayonet connector; and a tapered nipple.

35. The in-line tester of claim 24 wherein the colorimetric tester of the first chamber exhibits a colour change in response to the pH of a fluid in contact therewith.

36. The in-line tester of claim 24, wherein the colorimetric tester of the second chamber comprises a colorimetric capnometer, which exhibits a colour change in response to the concentration of carbon dioxide in contact therewith.

37. The in-line tester of claim 24, wherein either or both of the one-way valves comprise diaphragm check valves.

38. The in-line tester of claim 24, wherein the liquid-stop device comprises a porous or perforated element, which is at least partially manufactured from, or coated with, a hydrophobic material; or which has a hydrophobic layer on it.

39. The in-line tester of claim 38, wherein when the liquid-stop device is dry, gasses are able to pass through the pores or perforations therein, thus permitting the passage or transduction of gases/air through the liquid-stop device; but when the liquid-stop device is wetted by a liquid, the hydrophobicity of the liquid-stop device repels the liquid from its surface towards the pores/perforations, which are not hydrophobic, which results in liquid preferentially covering the liquid-stop device's pores/perforations, thereby preventing gas/air from passing through it.

40. The in-line tester of claim 24, wherein the main body comprises a set of stacked components, the stack comprising an upper component, a lower component and one or more intermediate components interposed between the upper and lower components, the fluid passageway being formed by any one or more of:
a recess formed in one component of the stack, the open part of the recess being closed by a surface of an adjacent component of the stack;
complementary recesses formed in adjacent components of the stack, the open parts of the recesses aligning so as to form the channel intersecting the interface between the adjacent layers; and
a through hole in one component of the stack, which aligns with a recess or surface of adjacent components of the stack on either side.

41. The in-line tester of claim 24, wherein the first and second chambers comprise windows, which permit visual inspection of the colorimetric testers.

42. The in-line tester of claim 41, wherein the window comprises the upper component of the stack being manufactured from a transparent material and by one or more chambers being formed by a cavity formed in an adjacent component of the stack, or by a through-aperture in a first adjacent component of the stack and by a surface aligning with the through-aperture second component of the stack adjacent the first adjacent component of the stack.
